(19) United States
(12) Patent Application Publication
(10) Pub. No.: US 2004/0254550 A1
(43) Pub. Date: Dec. 16, 2004

(54) Temperature change element for use in personal care products

(75) Inventors: Lei Huang, Duluth, GA (US); Cheryl A. Perkins, Appleton, WI (US); Jaeho Kim, Roswell, GA (US); Jeffrey E. Fish, Dacula, GA (US); Yanbin Huang, Roswell, GA (US)

Correspondence Address: KIMBERLY-CLARK WORLDWIDE, INC., 401 NORTH LAKE STREET, NEENAH, WI, 54956

(73) Assignee: Kimberly-Clark Worldwide, Inc.

(21) Appl. No.: 10/652,126

(22) Filed: Aug. 29, 2003

Related U.S. Application Data
(60) Provisional application No. 60/478,897, filed on Jun. 16, 2003.

(51) Int. Cl. A61F 13/15; A61F 13/20

(52) U.S. Cl. 604/361

(57) ABSTRACT

There is provided a temperature change element for use in personal care products where the temperature change element has a reactive component and a gel-forming agent. Upon exposure to liquids such as urine, the temperature change element will change in temperature substantially, thus letting the wearer know that he has urinated. The temperature change element may also have a binder to help keep the ingredients together and may have a colorant to further alert the user that the personal care product has been wetted

### BACKGROUND OF THE INVENTION

The present invention concerns temperature change elements for personal care products for the containment of bodily fluids. The personal care products are usually disposable.

Personal care products like diapers, training pants, adult incontinence products and the like are designed to move wastes, particularly liquid bodily wastes, away from the skin of the wearer in order to minimize skin irritation and discomfort. One unintended consequence of this movement is that the wearers, particularly small children, may not realize that the product has been soiled. This failure to feel discomfort and lack of realization of the signs of urination may be detrimental to the toilet training process in the case of children and so hamper the transition of the child from personal care product use to the wearing of normal clothing.

There remains a need for an indicator which will make it apparent to a wearer that the product has become soiled. Such an indicator would increase the awareness of the wearer of the signs of impending urination, thus allowing the user to seek toilet facilities.

### SUMMARY OF THE INVENTION

In response to the discussed difficulties and problems encountered in the prior art, a new indicator using a temperature change element has been developed. The temperature change element uses a component that reacts upon mixing with liquids such as urine. The temperature change element also has a gel-forming agent that serves to stabilize the reactant(s). Upon contact with liquids, the component reacts to make the temperature change element change in temperature while the gel-forming agent keeps the mixture from being too watery, while still avoiding being inflexible and rigid.

### BRIEF DESCRIPTION OF THE DRAWINGS

FIG. 1 is an illustration of a training pant suitable for use with the temperature change element.

FIG. 2 is a view of a training pant with the side seams opened and which has been substantially flattened for a better understanding of the relationship of the components.

FIG. 3 is a view of a hook and loop fastener.

FIG. 4 is a cross-sectional view of a laminate structure which may be used to contain the ingredients of the temperature change element.

FIG. 5 is a graph of the temperature change upon liquid insult of the temperature change element of Example 1. FIGS. 5 through 8 have a temperature scale of from 10to 40° C. on the vertical axis and a time scale in minutes from 0 to 30 on the horizontal axis.

FIG. 6 is a graph of the temperature change upon liquid insult of the temperature change element of Example 2.

FIG. 7 is a graph of the temperature change upon liquid insult of the temperature change element of Example 3.

FIG. 8 is a graph of the temperature change upon liquid insult of the temperature change element of Example 4.

FIG. 9 is a graph of the temperature change upon liquid insult of the temperature change element of Example 8.

FIG. 10 shows a perspective view of a diaper.

FIG. 11 shows a view of an absorbent underpants

FIG. 12 shows a view of an adult incontinence product.

### DETAILED DESCRIPTION OF THE INVENTION

The present invention is a temperature change element having within it a reactive mixture that reacts with liquids such as, for example, water and urine. This reaction is physical in nature by dissolution of a salt, and results in a change in temperature. This change in temperature, when it occurs in a personal care product, can alert the user that the product has been insulted (soiled). In order to facilitate the reaction, the temperature change element may be, for example, placed in a personal care product in an area adjacent to the urethra of the wearer so as to become wetted upon urination.

The temperature change element also contains a gel-forming agent to increase the viscosity of the resulting mixture so that the temperature change element is not too watery, i.e., so that it will not shift position easily and so be difficult to maintain in the position desired. The gel-forming agent serves to keep the source of the temperature change in place so that it does not wet the wearer's skin nor dissipate into the personal care product. The gel-forming agent also serves to suspend and distribute the reacting material.

While the discussion herein of the temperature change element of the invention generally revolves around training pants, the temperature change element is suitable for use not only in training pants but in other personal care products as well. Personal care products include but are not limited to diapers, training pants, absorbent underpants, adult incontinence products, and the like.

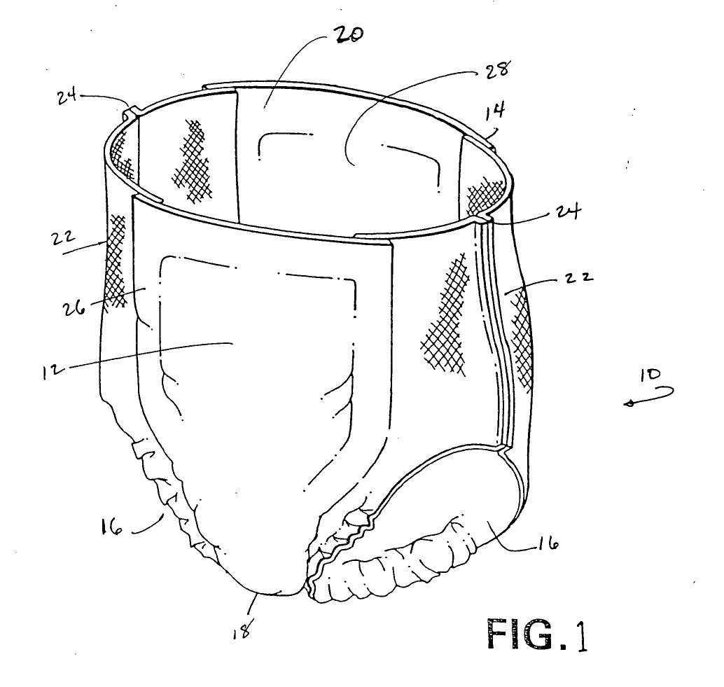

FIG. 1 shows a training pant 10 which has a front portion 12 and a rear portion 14 opposite the front portion 12. The pant 10 has leg openings 16 with a crotch portion 18 therebetween, as well as a waist opening 20. The pant 10 has two side portions 22 extending upward from the leg openings 16 and the side portions 22 may contain a seam or line of weakness 24 to allow for the easy removal of the pant 10 from the wearer by separating the seam 24. The toilet training pant 10 has an outer layer 26 which is generally though not necessarily totally impervious to the passage of liquids. The outer layer 26 is also referred to as a “backsheet”, “backing”, “outer cover”and “baffle”. The pant 10 has an inner, skin contacting surface or liner 28 which is desirably soft and non-irritating and usually has other layers between the outer layer 26 and liner 28.

The liner 28 is sometimes referred to as a bodyside liner or topsheet. In the thickness or Z-direction of the article, the liner material is the layer against the wearer's skin and so the first layer in contact with liquid or other exudate from the wearer. The liner further serves to isolate the wearer's skin from the liquids held in an absorbent structure and should be compliant, soft feeling and non-irritating.

Various materials can be used in forming the bodyside liner including but not limited to apertured plastic films, hydrogel films, woven fabrics, nonwoven webs, porous foams, reticulated foams and the like and the liner may be, for example, a laminate or multi-layer structure. Nonwoven materials have been found desirably suitable for use in forming the bodyside liner, including but not limited to spunbond or meltblown webs of polyolefin, polyester, polyamide (or other like fiber forming polymer) filaments, or bonded carded webs of natural polymers (for example, rayon or cotton fibers) and/or synthetic polymers (for example, polypropylene or polyester) fibers. For example, the bodyside liner can be a nonwoven spunbond web of synthetic polypropylene filaments. The nonwoven web can have a basis weight, for example, ranging from about 10.0 grams per square meter (gsm) to about 68.0 gsm, and more desirably from about 14.0 gsm to about 42.0 gsm. The liner 28 can be treated with a selected amount of surfactant, such as, for example, TRITON® X-102 surfactant or AHCOVEL® N-62 surfactant, or otherwise processed to impart a desired level of wettability and hydrophilicity.

The outer layer 26 is typically formed of a thin thermoplastic film, such as polyethylene film, which is substantially impermeable to liquid. The outer layer functions to prevent body exudates contained in a personal care product from wetting or soiling the wearer's clothing, bedding, or other materials contacting the article. The outer layer may be, for example, a polyethylene film having an initial thickness of from about 0.5 mil (0.012 millimeter) to about 5.0 mil (0.12 millimeter). The polymer film outer layer may be embossed and/or matte finished providing a more aesthetically pleasing appearance. Other alternative constructions for outer layer include but are not limited to woven or nonwoven fibrous webs that have been constructed or treated to impart the desired level of liquid impermeability, or laminates formed of a woven or nonwoven fabric and thermoplastic film.

The reactant used may be, for example, any of those known in the art as having a negative enthalpy of dissolution, including but not limited to those selected from a group of inorganic compounds including but not limited to ammonium nitrate, ammonium bromide, ammonium nitrite, ammonium sulfate, ammonium chloride, ammonium iodide, ammonium sulfamate, sodium chloride, sodium nitrate, sodium nitrite, sodium carbonate, sodium bicarbonate, potassium nitrate, potassium nitrite and organic compounds such as urea, methylurea, xylitol, ortho esters, menthone ketals and mixtures of these components. The reactant used hhhhhhhhhhhhhhhhhhhhhhhmay alternatively be, for example, any of those known in the art as having a positive enthalpy of dissolution, including but not limited to those selected from a group of compounds including aluminum sulfate, potassium aluminum sulfate or the like. The reactant should be present in an amount between about 50 and 99.9 weight percent on a dry basis of the temperature change element, more desirably between about 85 and 99 weight percent, still more desirably between about 90 and 97 weight percent.

The gel-forming agent used in the temperature change element may be organic or inorganic and serves to increase the viscosity of the temperature change element upon its being wetted. The increase in viscosity results in the temperature change element being too viscous to be carried by the flow of urine farther into the structure of the personal care product and thereby having its effectiveness diminished. In order to keep the temperature change element substantially in place, the viscosity of the wetted element should remain above 9600 centipoise, desirably above 20,000 centipoise.

Inorganic compounds such as metal oxides, metal alkoxides, or alkali metal salts of metal oxides can be used as the gel-forming agent. These include but are not limited to zinc oxide, tin oxide, titanium oxide, zirconium oxide, and silicates and aluminates in combination with solvents such as water and alcohols.

Useful organic gel-forming agents include but are not limited to organic compounds such as carbohydrates, including but not limited to starch; polyacrylamide; polyols such as pentaerythritol; or proteinaceous materials such as dried gelatin. These agents can form gels in combination with liquids such as water and urine. Further examples of organic- and inorganic-based gel systems are known to those skilled in the art.

Organic gel-forming agents also include but are not limited to a variety of polysaccharides including but not limited to starches. Starches, including but not limited to pre-gelatinized starches, may be modified by crosslinking to increase shear resistance and resistance to very high hydrogen-ion concentrations. Starches may be partially oxidized to yield improved stability. Starches can be derivatized by inorganic esterification with nitrates, sulfates, phosphates or xanthanates, or by organic esterification through treatment with carboxylic acids, acid anhydrides, acid chlorides, or vinyl esters. Starch ethers can also be formed for use in the present invention.

Gel-forming agents also include but are not limited to natural and modified natural polymers such as, for example, guar gum, gum tragacanth, locust bean gum, cellulosic materials, hydroxypropyl methylcellulose (e.g. Type F4M and Type K15MDGS from Dow Chemical Company, Midland, Mich. 48640) and hydroxyethyl cellulose.

Gel-forming agents further include but are not limited to superabsorbents. Superabsorbent material (SAM) generally refers to any substantially water-swellable, water-insoluble material capable of absorbing, swelling, or gelling, at least about 10 times its weight, and in some cases at least about 30 times its weight, in an aqueous solution, such as water. Moreover, a superabsorbent material can generally absorb at least about 20 grams of an aqueous solution per gram of the SAM, desirably at least about 50 grams, more desirably at least about 75 grams. One common superabsorbent material is, for example, FAVOR® 880 available from Stockhausen, Inc., of Greensboro, N.C.

One method of characterizing superabsorbents is by a test known as the absorption under tension or AUT test. This test is a modified version of TAPPI method T561pm-96 which is entitled “sorptive rate and capacity of bibulous paper products using gravimetric principles”. Appendix A2 of TAPPI method T561pm-96 discusses nonstandard variations. Superabsorbents suitable for the practice of this invention should have an AUT of at least 10 grams of liquid per gram of material.

In the AUT test, a specimen of the sample is place on a horizontal test plate such that its bottom surface rests on the plate and its upper surface is covered by a test weight. The sample is surrounded by a restraint such that it may only expand in one direction; the direction covered by the weight. The test plate is connected to a liquid reservoir by means of a siphon tube. The specimen is in contact with the effluent (8.5 g/l saline solution) of the siphon tube and the top surface of the liquid reservoir in relation to the sample may be adjusted during the test. The liquid reservoir is placed on a suitable weighing device. During the test, liquid is absorbed into the specimen and this absorption causes a reduction in the liquid present in the liquid reservoir which is measured by the weighing device. The decrease in weight in the liquid reservoir may be plotted directly or divided by the grams of the sample to provide an absorbent capacity per gram of sample over time.

Superabsorbents that are useful in the present invention can be chosen from classes based on chemical structure as well as physical form. These include but are not limited to superabsorbents with low gel strength, high gel strength, surface cross-linked superabsorbents, uniformly cross-linked superabsorbents, or superabsorbents with varied cross-link density throughout the structure. Superabsorbents may be, for example, based on chemistries that include but are not limited to poly(acrylic acid), poly(ethylene oxide), polystyrene sulphonate, hydroxylalkyl methacrylates, carboxy-methyl cellulose and poly(2-propenamide-co-2-propenoic acid, sodium salt). The superabsorbents may be in various forms, including but not limited to foams, macroporous or microporous particles or fibers, particles or fibers with fibrous or particulate coatings or morphology, ribbons, sheets or films. Superabsorbents may be in various length and diameter sizes and distributions. The superabsorbents may be in various degrees of neutralization and counter-ions are typically Li, Na, K, Ca. Superabsorbents which absorb liquids in the presence of a high concentration of ionic compounds are desirable in the practice of the invention since urine and the reactive mixture contain salt.

An exemplary superabsorbent was obtained from the Grain Processing Corporation of Muscatine, Iowa under the trade name WATER LOCK® G-400. This material is a homopolymer; poly(2-propenamide-co-2-propenoic acid, sodium salt). Additional types of superabsorbents not listed here which are commonly available and known to those skilled in the art as being relatively insensitive to salt concentration can also be useful in the present inventions.

The gel-forming agent should be present in an amount between about 0.1 and 50 weight percent on a dry basis of the temperature change element, more desirably between about 1 and 15 weight percent, still more desirably between about 3 and 10 weight percent.

Other optional ingredients may be added to the temperature change element if desired. These optional ingredients include but are not limited to colorants that may indicate the remaining useful life of the element, may indicate the temperature of the temperature change element, or may indicate that the element has been wetted and the reaction has been initiated. Another optional ingredient may be a binder that serves to increase the adherence of the components to one another and maintain the integrity of the temperature change element.

Colorants include but are not limited to FD&C Blue No. 1 (from BF Goodrich, 2235 Langdon Farm Road, Cincinnati, Ohio 45237) which changes from purple to blue after getting wet, FD&C Green No. 3 (from PYLAM Products Company, 2175 East Cedar Street, Tempe, Ariz. 85281) which changes from purple to green after getting wet and FD&C Yellow No. 6 (also from PYLAM Products Company), which changes from red to yellow after getting wet.

Binders which may be used in the practice of the invention are any which will bind the reactants and the gel-forming agent. Suitable binders include but are not limited to acrylic copolymers such as NAcrylic® salt sensitive binders from National Starch and Chemical Co. of Finderne, N.J., poly(vinylalcohol) (PVA), cationic poly(vinylpyrrolidone) copolymer (CPC) and polyvinylamine. Binders that are desirable do not lose their effectiveness upon being wetted by water and urine. The use of a binder can reduce the amount of gel-forming agent needed because the binder also increases the integrity of the reactive mixture by binding the ingredients together.

An effective amount of binder has been found to be between about 0.5 and 10 weight percent on a dry basis of the temperature change element, more desirably between 3 and 5 weight percent.

The temperature change element of this invention should reach a temperature of between about 5 and 15° C. above or below the normal skin temperature of about 35° C., or a minimum of 20° C. and a maximum of 50° C. after the liquid and the reactant come in contact. Temperatures below about 20° C. and above about 50° C. are undesirable since it is believed that damage to the skin of the wearer or pain may occur.

The temperature change element of this invention should maintain its temperature for at least about 10 minutes, more desirably at least about 20 minutes, and still more desirably more than 30 minutes. The life of the temperature change element is determined by the volume and type of reactant(s), as well as any insulation placed near the temperature change element.

Insulation can serve to moderate the temperature and extend the life of the temperature change element, and various insulating materials may, separately or in combination, be placed between the wearer and the temperature change element. These materials include but are not limited to nonwoven fabrics, woven fabrics, foams, coform materials, airlaid materials, bonded carded webs, films and combinations thereof and they may have a basis weight between about 10 and 200 gsm, more desirably between 25 and 150 gsm and still more desirably between 50 and 100 gsm. It is important that any insulating materials allow the passage of liquids to the temperature change element so that the reaction may occur.

A nonwoven fabric is a web having a structure of individual fibers or threads which are interlaid, but not in an identifiable manner as in a knitted fabric. Nonwoven fabrics or webs have been formed from many processes such as for example, meltblowing processes, spunbonding processes, and bonded carded web processes.

Spunbonded fibers refers to small diameter fibers which are formed by extruding molten thermoplastic material as filaments from a plurality of fine, usually circular capillaries of a spinneret with the diameter of the extruded filaments then being rapidly reduced as by, for example, in U.S. Pat. No. 4,340,563 to Appel et al., and U.S. Pat. No. 3,692,618 to Dorschner et al., U.S. Pat. No. 3,802,817 to Matsuki et al., U.S. Pat. Nos. 3,338,992 and 3,341,394 to Kinney, U.S. Pat. No. 3,502,763 to Hartman, and U.S. Pat. No. 3,542,615 to Dobo et al., the contents of which are hereby incorporated by reference as if set forth in their entirety. Spunbond fibers are generally not tacky when they are deposited onto a collecting surface. Spunbond fibers are generally continuous and have average diameters (from a sample of at least 10) larger than 7 microns, more desirably, between about 10 and 20 microns.

Meltblown fibers means fibers formed by extruding a molten thermoplastic material through a plurality of fine, usually circular, die capillaries as molten threads or filaments into converging high velocity, usually hot, gas (e.g. air) streams which attenuate the filaments of molten thermoplastic material to reduce their diameter, which may be to microfiber diameter. Thereafter, the meltblown fibers are carried by the high velocity gas stream and are deposited on a collecting surface to form a web of randomly dispersed meltblown fibers. Such a process is disclosed, for example, in U.S. Pat. 3,849,241 to Butin et al., the contents of which are hereby incorporated by reference as if set forth in their entirety. Meltblown fibers are microfibers which may be continuous or discontinuous, are generally smaller than 10 microns in average diameter, and are generally tacky when deposited onto a collecting surface.

Bonded carded webs are made from staple fibers which are sent through a combing or carding unit, which breaks apart and aligns the staple fibers in the machine direction to form a generally machine direction-oriented fibrous nonwoven web. Once the web is formed, it then is bonded by one or more of several known bonding methods such as powder bonding, pattern bonding, or through-air bonding.

Non-limiting examples of suitable foams include but are not limited to those produced by the General Foam Corporation of Paramus, New Jersey. Such foams are polyurethane foams under the trade designation “4000 Series”. Such foams are described in U.S. Pat. No. 4,761,324 to Rautenberg et al., the contents of which are hereby incorporated by reference as if set forth in their entirety.

Coforming is a process in which at least one meltblown diehead is arranged near a chute through which other materials are added to the web while it is forming. Such other materials may be, for example, pulp, superabsorbent particles, natural polymers (for example, rayon or cotton fibers) and/or synthetic polymers (for example, polypropylene or polyester) fibers, for example, where the fibers may be of staple length. Coform processes are shown in commonly assigned U.S. Pat. No. 4,818,464 to Lau and U.S. Pat. No. 4,100,324 to Anderson et al., the contents of which are hereby incorporated by reference as if set forth in their entirety. Webs produced by the coform process are generally referred to as coform materials.

Airlaid materials are formed by an airforming process in which bundles of small fibers having typical lengths ranging from about 3 to about 52 millimeters (mm) are separated and entrained in an air supply and then deposited onto a forming screen, usually with the assistance of a vacuum supply. The randomly deposited fibers then are bonded to one another using, for example, hot air or a spray adhesive. Examples of airlaying processes include but are not limited to the DanWeb process as described in U.S. Pat. No. 4,640,810 Laursen et al. and assigned to Scan Web of North America Inc, the Kroyer process as described in U.S. Pat. No. 4,494,278 Kroyer et al. and U.S. Pat. No. 5,527,171 Soerensen assigned to Niro Separation a/s, the method of U.S. Pat. No. 4,375,448 Appel et al. assigned to Kimberly-Clark Corporation, the contents of which are hereby incorporated by reference as if set forth in their entirety.

In one aspect of the invention, the reactant useful in this invention may be coated with the gel-forming agent and may use a binding agent or binder to hold the two together. Coating of the reactant by the gel-forming agent may be accomplished by mixing the three ingredients together. This may be followed by freeze drying to remove moisture from the resultant matrix as discussed below. Freeze drying is a desirable method of forming the temperature change element of the invention, but is not the only method. Room temperature or elevated temperature, such as drying in an oven, may also be used though the quality of the product has been found to be less than that produced by freeze drying, which yields a more porous structure. A more porous structure is desired because it allows liquid to flow into the structure to come in contact with the reactive mixture.

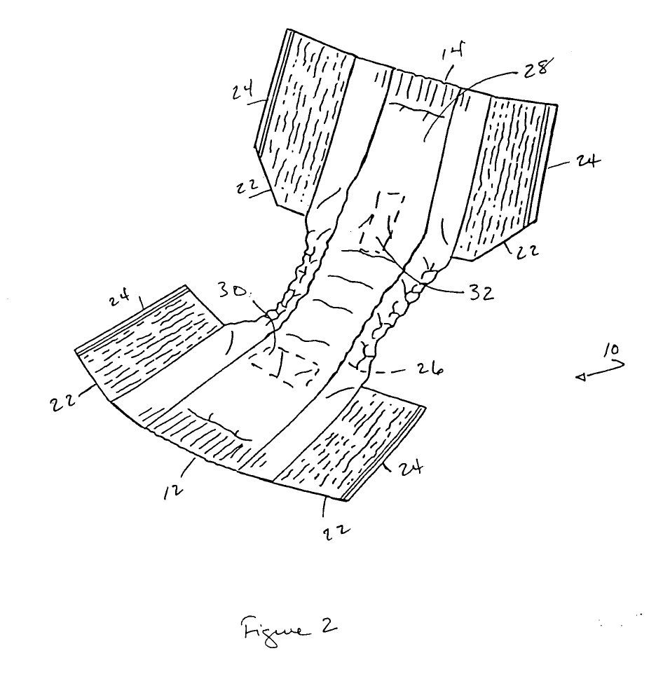

FIG. 2 illustrates a training pant 10 in an open, substantially flat configuration wherein the seams 24 of the side portions 22 have been separated. The liner 28 is visible with the outer layer 26 below and the front portion 12 and rear portion 14 indicated. The temperature change element 30 may be placed in the pant 10 as illustrated by the dashed lines. The temperature change element 30 may be placed anywhere within the pant 10 though desirably below the liner 28 in an area aligned to be near the urethra of the wearer. The temperature change element 30 shown is a rectangular shape aligned across the training pant in a transverse direction.

The location of the temperature change element may differ between the genders as will be appreciated by those skilled in the art, or the temperature change element may be oriented longitudinally, for example, for use with either gender. Alternatively, multiple temperature change elements may be used for training pants to be used by both genders. Furthermore, a second temperature change element 32 may be placed in an area aligned to be near the anus of the wearer to indicate the discharge of feces containing liquid.

FIG. 2 illustrates the temperature change element 30 as a generally elongated or rectangularly shaped area though it should be appreciated that other shapes and orientations may be used. (Alternative shapes will be discussed further below.) The area for accepting the temperature change element 30 of FIG. 2 may be formed by creating a pocket within the liner 28 of the pant 10 where the liner 28 is a multi-layer structure by, for example, attaching three sides of a generally rectangular area, inserting the temperature change element 30 as a mixture between the layers, and closing the remaining side of the area. The area of the temperature change element is a matter of design preference and may be large or small in relation to the size of the personal care product, provided, however, that the temperature limits discussed herein are observed.

Alternatively, the temperature change element 30 may be pre-packaged in, for example, a multi-layer or bag-like configuration and inserted within the pocket. The temperature change element may be packaged as a bag by using a liquid permeable material such as a superabsorbent film or a nonwoven web which may be folded and the seams or edges sealed to retain the temperature change element ingredients.

The temperature change element 30, if in the form of a pre-packaged configuration, may also be attached directly onto the surface of the liner 28 without the use of a pocket, either adhesively, using ultrasonic bonding, with tape or through the use of hook and loop fasteners where either the hook or loop is adhered to the liner and the other component attached to the temperature change element.

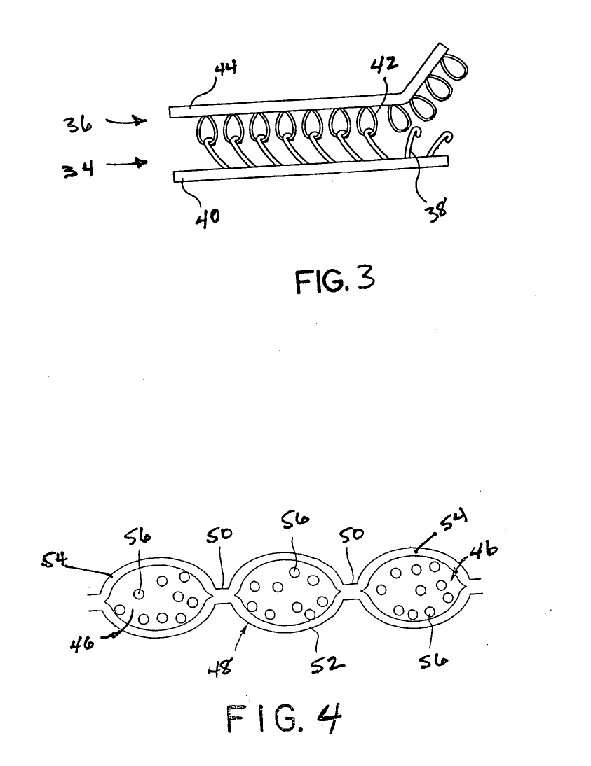

The use of a releasably attached temperature change element 30 allows the parent or caregiver to move or remove the temperature change element if desired. As shown in FIG. 3, a hook component 34 and a loop component 36 can be brought together to be releasably attached to one another. The hook component 34 has a number of individual hooks 38 protruding generally perpendicularly from a resilient hook backing material 40. Similarly, the loop component 36 has a number of individual loops 42 protruding generally perpendicularly from a resilient loop backing material 44. The individual hooks 38 and the individual loops 42, when brought into contact with one another, engage with one another, with the hooks 38 latching onto the loops 42, until forcibly separated, thereby pulling the hooks 38 out of the loops 42. Further discussion and illustration of hook and loop fasteners may be found, for example, in U.S. Pat. No. 4,794,028 to Fischer, the contents of which are hereby incorporated by reference as if set forth in their entirety.

Another useful form of packaging the materials of this invention is by the use of laminates having enclosed discrete regions of material as described in U.S. application Ser. No. 10/027,787, the contents of which are hereby incorporated by reference as if set forth in their entirety. This application teaches the making of a laminate having pockets in a fabric as shown in FIG. 4. Using this form of packaging reduces the problem associated with the movement of materials associated with larger packages and more evenly distributes the cooling or heating effect.

Referring to FIG. 4 which is a cross-sectional view of a laminate 48; reactant and gel-forming particles 56, for example, can be initially deposited onto a first substrate 52. Once deposited, a second substrate 54 can then be bonded to portions of the first substrate 52 in a number of ways such as hydroentanglement, needling, ultrasonic bonding, adhesive bonding, stitchbonding, through-air bonding and thermal bonding. The substrates are generally bonded together only at those portions on which the discrete regions of particles have not been deposited. As a result, discrete regions of particles 56 can be contained within unfused portions or pockets 46 within a laminate 48. In addition, the fused portions 50 of the laminate 48 can be formed from certain materials, such as films, hydrogel films or nonwoven webs, which become substantially impermeable to liquids when fused together. The unfused portions of the substrates can remain substantially permeable to liquids such that any liquid contacting the laminate structure is primarily directed or transported to the pockets 46 of the laminate structure so that they contact the temperature change element.

The pockets 46 of the laminate structure formed such as described above, can vary in size and shape. For example, the pockets can have regular or irregular shapes. Some regular shapes can include but are not limited to, for example, circles, ovals, ellipses, squares, hexagons, rectangles, hourglass-shaped, tube-shaped, etc. Moreover, in some instances, some pockets of the laminate structure may have different shapes and/or sizes than other pockets.

Regardless of the particular shape utilized, the pockets are generally formed to be relatively small in size so that they do not substantially inhibit the flexibility of the resulting laminate structure. The approximate width “w” to height “h” ratio of the pockets 46 (i.e., w/h) can be, for example, less than 10, desirably between about 1 to about 8, and more desirably, between 1 to about 5. The approximate height “h” can be equal to less than about 2.5 cm, in some embodiments less than about 1.25 cm, and in some embodiments, between about 0.013 cm and about 1.2 cm. In addition, the spacing between the pockets can be varied. The approximate distance that the pockets 46 are spaced apart can, in some embodiments, be greater than about 0.16 cm.

In yet another configuration, a superabsorbent film may be coated with the non-water reactant. The film protects the skin from exposure to possibly irritating materials like ammonium nitrate and also immobilizes the reactant. It is further possible to use a non-irritating reactant like xylitol on the film, further reducing the likelihood of damage to the skin.

The size of the temperature change element and the amount of reactant used will depend on the size of the personal care product as well as the characteristics of the reactant, though it's believed that the amount of reactant used should provide an energy flux of between about 6 and 30 cal/cm², as described in U.S. Pat. No. 5,681,298, hereby incorporated by reference. This energy flux will generally result in a range for the reactants per unit area of the personal care product in the range of from 0.1 to 0.5 g/cm², desirably 0.2 to 0.4 g/cm².

Exemplary ranges for the reactants may be given and the amount of other ingredients calculated as above. Ammonium nitrate, for example, may be used in a personal care product in an amount between about 1 and 50 grams, desirably between about 1 and 10 grams and more desirably between 1 and 3 grams. Urea may be used in a personal care product in an amount between about 1 and 50 grams, desirably between about 1 and 10 grams and more desirably between 2 and 5 grams. Xylitol may be used in a personal care product in an amount between about 1 and 50 grams, desirably between about 1 and 20 grams and more desirably between 5 and 10 grams.

In use, liquid, e.g., urine, will enter the indicator through the liquid permeable liner or other material that surrounds the temperature change element ingredients, and travel away from the point of deposit. As this occurs the gel-forming agent will swell as it absorbs liquid and the reactant will simultaneously cool or heat as it reacts with liquid. The swelling of the gel-forming agent substantially prevents the reactant from being carried away by the flowing urine, even if the reactants are surrounded by a liquid permeable material, i.e., the gel-forming agent prevents the temperature change element from being too watery.

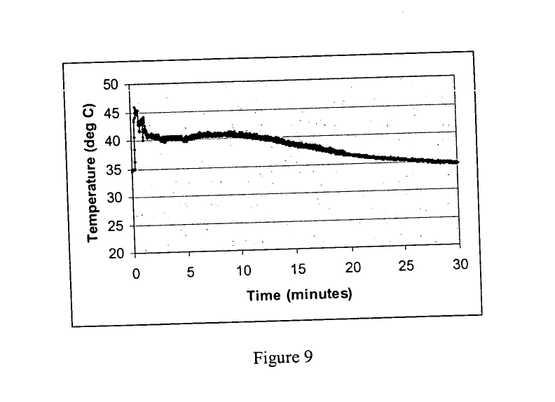

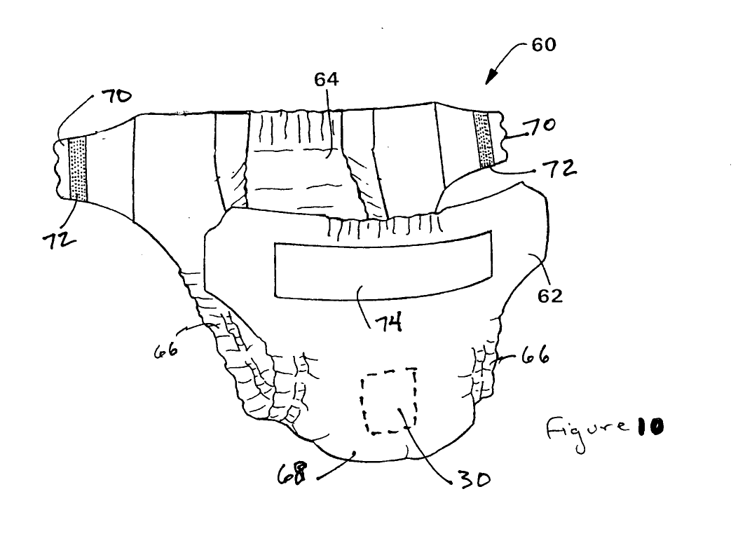

As mentioned above, the temperature change element of the invention may be used in personal care products not limited to training pants. Other suitable personal care products include but are not limited to those illustrated in FIGS. 9 through 11 and described in, for example, U.S. Pat. Nos. 5,192,606; 5,906,306; 5,776,123 and 5,649,916, the contents of which are hereby incorporated by reference as if set forth in their entirety. These personal care products include diapers, training pants, absorbent underpants, adult incontinence products and the like.

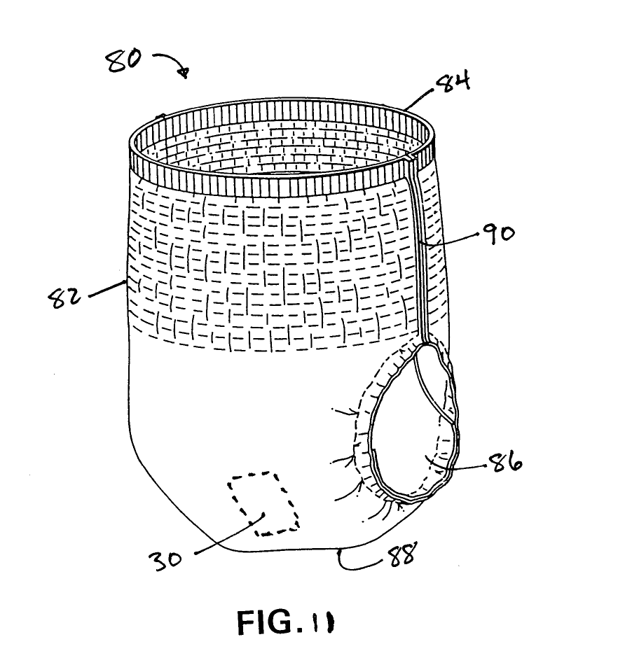

FIG. 10 shows a perspective view of a diaper 60 having a front portion 62 and a rear portion 64 opposite the front portion 62. The pant 60 has leg openings 66 with a crotch portion 68 therebetween. The diaper 60 has refastenable extending members 70 on either side of the rear portion 64 used to fasten to the front portion 62 when the diaper is in use on a child. The extending members 70 may have, for example, hooks 72 disposed thereon to entangle with loops 74 located on the front portion 62 of the diaper 60. The temperature change element 30 is shown in position for a male child.

FIG. 11 shows a view of an absorbent underpant 80 with a front portion 82, rear portion 84, leg openings 86 with a crotch portion 88 therebetween. The front portion 82 and rear portion 84 are connected by seams 90 on either side. The temperature change element 30 is shown in dashed lines in the underpant 80.

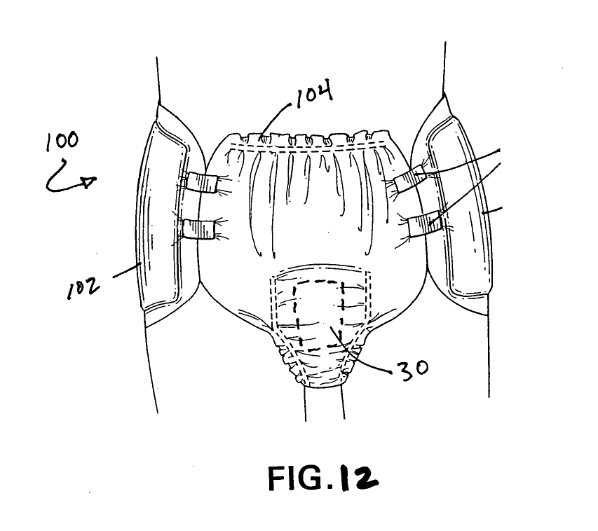

FIG. 12 shows a view of an adult incontinence product 100 in position on a wearer. The incontinence product has visible sides 102 and a front portion 104, connected by straps 106 on either side of the front portion 104. The temperature change element 30 is shown in dashed lines in the incontinence product 100.

### EXAMPLE 1

In this example, 25 grams of an endothermic reactant, in this case ammonium nitrate pellets from the Aldrich Chem. Co. of Milwaukee, Wis., was mixed with 10 grams of WATER LOCK® G-400 superabsorbent material, which is a homopolymer; poly(2-propenamide-co-2-propenoic acid, sodium salt) powder. Also included in this mixture was 1 ml of NAcrylic® SSB-66 liquid salt sensitive polymer binder, available from National Starch and Chemical Corporation of Bridgewater, N.J. and 99 ml of deionized water. This mixture was spread on a glass Petri dish and dried by freeze drying at −46° C. for 8 hours, resulting in a porous matrix, sheet or material with a sponge-like appearance. The freeze drying used a Freezone® 4.5 Liter Benchtop Model 77500 from Labconco, of 8811 Prospect, Kansas City, Mo. The vacuum condition was at 133×10⁻³ mBAR.

Three standard OMEGA® K-type thermocouples (Omega Engineering Inc., One Omega Drive, Stamford, Conn.) were placed immediately below the surface of a 10 gram porous sheet produced above. The sheet was placed on a metal heating plate having a temperature maintained at 35° C. In a room under standard atmospheric temperature and pressure and allowed to stabilize. A test was performed by rapidly pouring 50 ml of a 35° C. saline solution onto the sheet and most of the saline solution flowed into the porous matrix. The thermocouple temperatures were recorded and averaged using LABTECH® V12 data acquisition software from Labtexh of Two Dundee Park, Suite 109, Andover Mass., in order to develop the graph of FIG. 5. Shortly after delivery of the liquid, the material became cold to the touch, indicating that the endothermic reactants were dissolving and acting as a heat sink. The materials remained cool to the touch for about 20 minutes after liquid delivery. The viscosity of the mixture was also measured, using a Brookfield Viscometer LV model (Brookfield Engineering Laboratories, Inc., 11 Commerce Boulevard, Middleboro, Mass.) with a #4 spindle at 6 rpm, and was found to be 62,000 centipoise.

### EXAMPLE 2

A porous matrix was made by slowly moisturizing ammonium nitrate pellets (Aldrich Chem.) by spraying them with a light water mist totaling about 1 ml of water, and then thoroughly mixing the moistened pellets with salt-insensitive superabsorbent poly(2-propenamide-co-2-propenoic acid, sodium salt) powder. No binder was used. A mixture of 25 grams of moistened pellets and 10 grams of superabsorbent was gently pressed into a sheet form and dried at about 22° C. (room temperature) for 24 hours at standard humidity (approx. 70 percent).

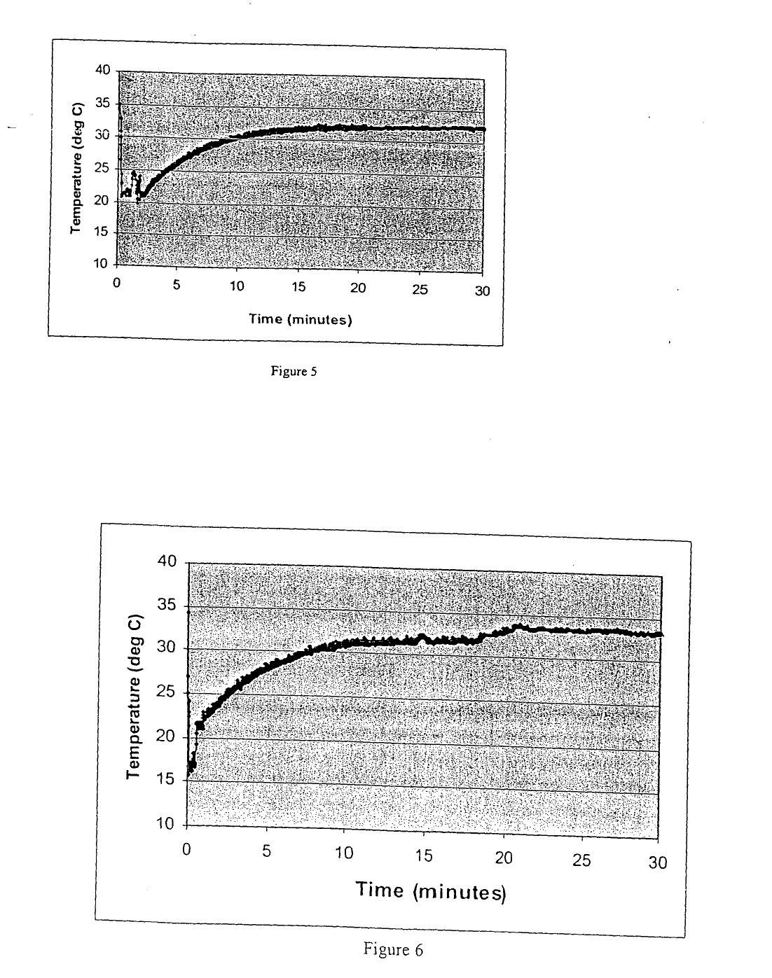

A 10 gram sample of the sheet was rapidly insulted with 50 ml of 35° C., 1 percent saline solution in the same manner as Example 1 and the temperature recorded and averaged as in Example 1 to develop the graph of FIG. 6. The sheet remained cool to the touch for at least 20 minutes.

### EXAMPLE 3

A training pant was modified to include a temperature change element. A rectangular 3.25 cm by 7.25 cm pocket was formed in a spunbond nonwoven training pant laminate liner in the transverse direction by heat sealing three sides of the pocket. A mixture of 10 grams of crystalline Xylitol C powder (Danisco USA Inc. of Ardsley, N.Y.) and 5 weight percent WATER LOCK® G-400 superabsorbent material was placed in the pocket. Three standard OMEGA® K-type thermocouples were placed immediately below the liner near the ingredients. The modified training pant was placed on a metal heating plate having a temperature maintained at 35° C. and allowed to stabilize. The modified training pant was rapidly insulted with 100 ml of saline solution at 35° C. The temperatures were recorded and averaged as in Example 1 to develop the graph of FIG. 7. The average temperature dropped from 35° C. to 20° C. within a minute and the cooling effect lasted about 8 minutes.

### EXAMPLE 4

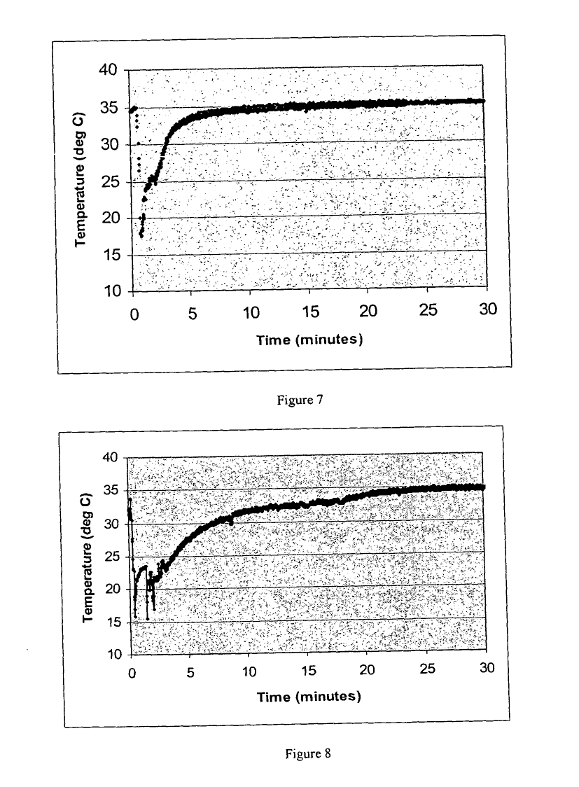

A training pant was modified as in Example 3. A mixture of 10 grams of 98% urea powder (Aldrich Chemical, catalog no. 20,8884) and 5 weight percent WATER LOCK® G-400 superabsorbent material was placed in the pocket. Three standard OMEGA® K-type thermocouples were placed and the modified training pant tested in the same manner as in Example 3. The temperatures were recorded and averaged as in Example 1 to develop the graph of FIG. 8. The average temperature dropped from 35° C. to 15° C. within a minute and the cooling effect lasted about 20 minutes.

### EXAMPLE 5

A temperature change element was made by depositing onto a substrate a mixture of 10 weight percent ammonium nitrate pellets from the Aldrich Chem. Co. and the balance superabsorbent. A second substrate layer was then laminated onto the first in a pattern as shown in FIG. 3. The substrate layers were apertured elastomeric meltblown nonwoven fabrics having a basis weight of 35 grams per square meter (gsm). The meltblown fabric was made from copolyetherester from Kraton Polymers Inc. of Houston, Tex. The aperturing pattern for the meltblown layers was an expanded Hansen Pennings or “EHP” bond pattern which produces a 16% bond area with a square pin having a side dimension of 0.037 inches (0.94 mm), a pin spacing of 0.097 inches (2.464 mm) and a depth of 0.039 inches (0.991 mm). Formation of an elastomeric nonwoven web from polyester elastomeric materials is disclosed in, for example, U.S. Pat. No. 4,741,949 to Morman et al. and U.S. Pat. No. 4,707,398 to Boggs, hereby incorporated by reference.

Upon insult with saline solution, the superabsorbent began to swell and the temperature to drop as measured by touch, indicating successful absorption of the saline solution.

### EXAMPLE 6

Endothermic salts (NH₄NO₃) pellets (Aldrich Chem. Co.) In the amount of 25 grams were placed in a 3.25 cm×7.25 cm bag made from absorbent polyurethane foam (1.0 mm thick Rynel 562-B Polyurethane absorbent foam from Rynel, Route 27, Boothbay, Me.). Upon insult by saline solution, the bag began to swell and cool to the touch. This indicates that the bag successfully allowed the passage of liquid to the internal area where the endothermic salts were located.

### EXAMPLE 7

Endothermic salts were loaded into a gel film by making the hydrogel film in the presence of endothermic salts. A solution was made with 5 grams of urea (Aldrich Chem. Co.) was mixed with 4.10 g of distilled PEGMA 400 (Poly(ethylene glycol) methacrylate (Aldrich Chem. Co.), 0.11 g of TEGDMA (tetraethyleneglycol dimethacrylate from Aldrich Chem. Co. as the crosslinker) and 0.7458 g of Pluronic® 25R2 (from BASF Corporation, Mount Olive, N.J. as the surfactant). The gel formation was initiated by adding 0.05 grams of initiator (NH₄)₂S₂O₈ and putting the system in an 80° C. oven for 2 hours. A translucent flexible gel was formed and then freeze dried as in Example 1. The final product was a white film about 1.6 mm thick. The rigidity can be adjusted by the dryness and a flexible gel film can be achieved by adding a small amount of water. Upon rapid insult with 25 ml of saline solution, the gel film began to swell and cool to the touch. This indicates that the gel film successfully absorbed the liquid and dissolved the loaded endothermic salts.

### EXAMPLE 8

A training pant was modified to include a temperature change element. A rectangular 3.25 cm by 7.25 cm pocket was formed in a spunbond nonwoven training pant laminate liner in the transverse direction by heat sealing three sides of the pocket. A mixture of 20 grams of sodium thiosulfate (99%, from Aldrich Chem. Co.) and 5 weight percent WATER LOCK® G-400 superabsorbent material was placed in the pocket. Three standard OMEGA® K-type thermocouples were placed immediately below the liner near the ingredients. The modified training pant was placed on a metal heating plate having a temperature maintained at 35° C. and allowed to stabilize. The modified training pant was rapidly insulted with 100 ml of saline solution at 35° C. The temperatures were recorded and averaged as in Example 1 to develop the graph of FIG. 9. The average temperature rose from 35° C. to 45° C. within a minute and the heating effect lasted about 15 minutes.

As will be appreciated by those skilled in the art, changes and variations to the invention are considered to be within the ability of those skilled in the art. Examples of such changes are contained in the patents identified above, each of which is incorporated herein by reference in its entirety to the extent it is consistent with this specification. Such changes and variations are intended by the inventors to be within the scope of the invention.

What is claimed is:

1) A temperature change element for use in personal care products comprising at least one gel-forming agent and a reactant that reacts with liquid to produce a temperature of between about 5 and 15° C. different from normal skin temperature.

2) The temperature change element of claim 1 wherein said reactant comprises a compound selected from the group comprising ammonium nitrate, ammonium bromide, ammonium nitrite, ammonium sulfate, ammonium chloride, ammonium iodide, ammonium sulfamate, sodium chloride, sodium nitrate, sodium nitrite, sodium carbonate, sodium bicarbonate, potassium nitrate, potassium nitrite, xylitol, urea, methylurea, ortho esters, menthone ketals and mixtures thereof.

3) The temperature change element of claim 2 wherein said temperature is between about 15 to 25° C. after about 1 to 5 minutes after said liquid and said reactant come in contact.

4) The temperature change element of claim 2 wherein said temperature change element reaches a temperature of between about 20 and 30° C.

5) The temperature change element of claim 3 wherein said temperature is maintained for at least about 10 minutes.

6) The temperature change element of claim 3 wherein said temperature is maintained 20 for at least about 20 minutes.

7) The temperature change element of claim 3 wherein said temperature is maintained for at least about 30 minutes.

8) The temperature change element of claim 1 comprising a mixture of an endothermic reactant in the amount of between about 50 and 99.1 weight percent and a gel-forming agent in the amount of between about 0.1 and 50 weight percent, which has a viscosity of at least 9600 centipoise after reaction with liquid.

9) The temperature change element of claim 1 comprising a mixture of an endothermic reactant in the amount of between about 85 and 99 weight percent and a gel-forming agent in the amount of between about 1 and 15 weight percent.

10) The temperature change element of claim 1 comprising a mixture of an endothermic reactant in the amount of between about 90 and 97 weight percent and a gel-forming agent in the amount of between about 3 and 10 weight percent.

11) The temperature change element of claim 8 further comprising a binder in the amount of about 0.5 and 10 weight percent.

12) A temperature change element for use in personal care products comprising a superabsorbent, a binder and a reactant that reacts with liquid to produce a temperature of between about 5 and 15° C. different from normal skin temperature.

13) The temperature change element of claim 12 which is disposed in a personal care product having a liner and an outer cover, wherein said element is between said liner and said outer cover in an area aligned to be near a wearer's urethra.

14) The temperature change element of claim 13 which is disposed on said liner, from which it may be releasably attached.

15) The temperature change element of claim 14 wherein said temperature change element is releasably attached by a hook and loop fastener.

16) The temperature change element of claim 13 wherein said personal care product further has a pocket in which said temperature change element may be disposed.

17) The temperature change element of claim 13 wherein said personal care product further comprises a layer of insulation disposed on said temperature change element on a side toward said wearer.

18) The temperature change element of claim 17 wherein said layer of insulation is made from a material selected from the group consisting of woven fabrics spunbond fabrics, meltblown fabrics, bonded carded webs, coform webs, airlaid webs, films, foams and combinations thereof and has a basis weight between about 10 and 200 gsm.

19) The temperature change element of claim 18 wherein said material has a basis weight between 25 and 150 gsm.

20) The temperature change element of claim 18 wherein said material has a basis weight between 50 and 100 gsm.

21) The temperature change element of claim 13 further comprising a colorant that indicates that the element has been wetted.

22) The temperature change element of claim 13 disposed in a personal care product, further comprising a second temperature change element placed in said personal care product in an area aligned to be near a wearer's anus to indicate a discharge of feces containing a large amount of liquid.

23) The temperature change element of claim 13 wherein said personal care product in which said element is disposed is selected from the group consisting of diapers, training pants, absorbent underpants and adult incontinence products.

24) A training pant comprising a liner and an outer cover, and a temperature change element comprising a superabsorbent and a reactant that reacts with liquid to produce a temperature of between about 5 and 15° C. different from normal skin temperature.

25) The training pant of claim 24 wherein said temperature change element further comprises a binder.

26) The training pant of claim 25 wherein said temperature change element is disposed between said liner and said outer cover.

27) The training pant of claim 25 further comprising a pocket in which said temperature change element may be disposed.

28) The training pant of claim 25 wherein said temperature change element is releasably attached to said liner.

29) The training pant of claim 25 further comprising a layer of insulation between said temperature change element and a wearer.

30) The training pant of claim 25 wherein said temperature change element reaches a temperature of between about 15 to 25° C. after about 1 to 5 minutes after said liquid and said reactant come in contact.

31) The training pant of claim 25 wherein said temperature change element reaches a temperature of between about 20 and 30° C.

32) The training pant of claim 24 wherein said reactant comprises a compound selected from the group comprising ammonium nitrate, ammonium bromide, ammonium nitrite, ammonium sulfate, ammonium chloride, ammonium iodide, ammonium sulfamate, sodium chloride, sodium nitrate, sodium nitrite, sodium carbonate, sodium bicarbonate, potassium nitrate, potassium nitrite, xylitol, urea, methylurea, ortho esters, menthone ketals and mixtures thereof.

33) The training pant of claim 24 wherein said superabsorbent is poly(acrylic acid), poly(ethylene oxide), polystyrene sulphonate, hydroxylalkyl methacrylates, carboxy-methyl cellulose and poly(2-propenamide-co-2-propenoic acid, sodium salt).

34) The training pant of claim 25 comprising a mixture of an endothermic reactant in the amount of between about 50 and 99.1 weight percent, superabsorbent in the amount of between about 0.1 and 50 weight percent and binder in the amount of about 0.5 and 10 weight percent.

35) The training pant of claim 25 comprising a mixture of an endothermic reactant in the amount of between about 85 and 99 weight percent, superabsorbent in the amount of between about 1 and 15 weight percent and binder in the amount of about 0.5 and 10 weight percent.

36) The training pant of claim 25 comprising a mixture of an endothermic reactant in the amount of between about 90 and 97 weight percent, superabsorbent in the amount of between about 3 and 10 weight percent, and binder in the amount of about 0.5 and 10 weight percent.

37) A training pant comprising a liner, an outer cover and a laminate between said liner and said outer cover, said laminate comprising a plurality of fused portions and a plurality of unfused portions, said unfused portions containing a gel-forming agent and a reactant, said fused portions directing liquid to said unfused portions and said gel-forming agent and said reactant.
